(19) United States
(12) Patent Application Publication
(10) Pub. No.: US 2014/0273031 A1
(43) Pub. Date: Sep. 18, 2014

(54) IN-VITRO METHOD FOR MONOCLONAL ANTIBODY PRODUCTION

(71) Applicant: THE CLEVELAND CLINIC FOUNDATION, Cleveland, OH (US)

(72) Inventors: Earl J. Poptic, North Royalton, OH (US); Xiaoxia Li, Cleveland, OH (US)

(73) Assignee: THE CLEVELAND CLINIC FOUNDATION, Cleveland, OH (US)

(21) Appl. No.: 14/212,688

(22) Filed: Mar. 14, 2014

Related U.S. Application Data
(60) Provisional application No. 61/793,646, filed on Mar. 15, 2013.

(51) Int. Cl. C12N 5/16; C12N 15/87

(52) U.S. Cl. CPC C12N 5/163; C12N 15/87
USPC 435/7.92; 435/289.1

(57) ABSTRACT

A method of producing an IgG type monoclonal antibody in vitro is described. The method includes the steps of: (A) providing B-cells obtained from a transgenic non-human mammal whose genome comprises a disruption of an Act1 gene; (B) contacting the B cells with an antigen under conditions which result in formation of an antibody specific to the antigen by the B cells; (C) fusing the B cells with one or more immortal cells, to produce one or more hybridomas which express IgG type monoclonal antibodies that specifically bind to the antigen; (D) selecting a hybridoma which expresses an antibody specific to the antigen; and (E) obtaining monoclonal antibody produced by the selected hybridoma. Kits for carrying out the method of producing an IgG type monoclonal antibody are also described.

### CROSS-REFERENCE TO RELATED APPLICATIONS

This application claims priority to U.S. Provisional Patent Application No. 61/793,646, filed Mar. 15, 2013, which is incorporated herein by reference in its entirety.

### BACKGROUND

Monoclonal antibodies are reagents that are utilized widely in both the academic and commercial pharmaceutical and biotechnology sectors. They are used to detect specific antigens that are commonly found on proteins or peptides, through an interaction with a specific site on the antigen known as an epitope. The same epitope may be found on different proteins. Likewise, any one protein may display several different epitopes. Such reagents are commonly used in basic research, diagnostic, assay development and more recently therapeutic applications.

Although well established and widely utilized, the standard method for producing monoclonal antibodies by fusing splenic lymphocytes with myeloma cells has remained largely unmodified for the past 25 years. The standard method involves forming hybrid cell lines (called hybridomas) by fusing a specific antibody-producing B cell with a myeloma (B cell cancer) cell that is selected for its ability to grow in tissue culture, and was developed by developed by Köhler and Milstein in 1975. Due to some of the limitations inherent in this technique, many new technologies have been developed to produce antigen recognition reagents with similar characteristics—and in some cases structures—to the traditional monoclonal antibody. These include phage display, phagemid display, single-chain variable fragment (scFV) and others. Despite the potential advantages and promise of many of these new technologies, antibodies produced by the standard method, or a simple modification thereof, remains the method of choice in most in-house and contract laboratories.

Monoclonal antibodies are typically made using an expensive process of fusing myeloma cells with the spleen B cells from a mouse that has been immunized for 2-3 months with multiple injections of relatively large quantities of the desired antigen. An ideal approach for the production of a human monoclonal antibody would circumvent these laborious methods and procedures. In addition, current in vitro methods using normal mouse spleens produce predominantly IgM monoclonal antibodies; however, IgG is the preferred isotype for monoclonal antibodies as IgM monoclonal antibodies are generally less soluble, more likely to precipitate, more difficult to purify and can exhibit increased high non-specific binding. In addition, IgGs can be used in various immunoassays because of the availability of secondary agents, whereas IgMs in general, are not.

One approach to improve monoclonal antibody production would be to modify B cell biochemistry through genetic modification. The adaptor molecule Act 1 has been identified as an important regulator in signaling pathways mediated by CD40 and BAFF, which are involved in B cell survival and differentiation. Endogenous Act1 is recruited to CD40 in B cells upon stimulation with CD40 ligand (CD40L). Act-1 deficient mice display major lymphoid system defects, which is marked with lymphadenopathy, splenomegaly, hypergammaglobulinemia, inflammation in multiple tissues and the production of autoantibodies. An increase in the numbers of peripheral B cells coupled with an increase in CD40- and BAFFR-mediated B cell survival in Act-1-deficient mice indicates that Act1 is an important modulator in humoral immune responses by regulating CD40 and BAFFR signaling in B cells.

The constitutive levels of IgG are substantially increased, more than 10-fold, in Act-1 deficient mice compared to wild type controls. When challenged in vivo by the T-cell dependent antigen nitro-phenol-conjugated chicken γ-globulin (NP₂₈-CGG), Act1-deficient mice developed much higher titers of both total and high-affinity antigen specific IgG2b antibodies and just slightly increased total IgG antibodies but significantly increased high-affinity IgG1 antibodies. However, IgM antigen-specific antibody production was unchanged. These results were similar in Act-1 deficient mice immunized with T cell-independent antigen NP-Ficoll where Act-1 deficient mice developed much higher titers of total NP-specific IGG2a and IgG2b antibodies. However, NP-specific IgM and IgG1 production was unchanged (Qian et al., Immunity 21:575-587 (2004)).

### SUMMARY

The present invention relates to an in vitro method for the production of monoclonal antibodies, in particular of IgG type, making use of Act1 deficient B cells.

Recent data show that an Act 1 deficient mouse spleen cell exposed to an antigen in vitro can be fused to an immortal myeloma cell, thereby producing a hybridoma capable of expressing antigen specific IgG monoclonal antibodies.

Accordingly, in one aspect, the present invention provides a method of producing an IgG type monoclonal antibody. The method includes the steps of: (A) providing B-cells obtained from a transgenic non-human mammal whose genome comprises a disruption of an Act1 gene; (B) contacting the B cells with an antigen under conditions which result in formation of an antibody specific to the antigen by the B cells; (C) fusing the B cells with one or more immortal cells, to produce one or more hybridomas which express IgG type monoclonal antibodies that specifically bind to the antigen; (D) selecting a hybridoma which expresses an antibody specific to the antigen; and (E) obtaining monoclonal antibody produced by the selected hybridoma.

In some embodiments, the B cells are present in a spleen cell isolate. In additional embodiments, the method further includes the step of isolating the B cells from the transgenic non-human mammal.

In some embodiments, the monoclonal antibodies are humanized monoclonal antibodies. In certain embodiments, the mammal is a mouse.

In certain embodiments, the disruption of an Act 1 gene includes a homozygous disruption of an Act 1 gene. In some embodiments, the disruption of an Act 1 gene can also include a deletion of exon 2 of the Act1 gene.

In some embodiments, the step of selecting a hybridoma includes the use of an immunoassay. The immunoassay can include an ELISA assay.

In some embodiments, the step of fusing the B cells with an immortal cell includes the use of a PEG polymer complex. The immortal cell can include a mouse myeloma cell. In some embodiments, the mouse myeloma cell is a SP2/0 mouse myeloma cell.

In additional embodiments the antigen contacted with the B cells is selected from the group consisting of human pathogens, allergens, bacteria, toxins, mycoplasma, fungi and viruses. In certain embodiments, the step of contacting the B cells with the antigen includes administering HCS (hybridoma cloning supplement) to the B cells.

Another aspect of the present invention provides a kit for producing an IgG monoclonal antibody specifically directed to an antigen in vitro. The kit includes: (A) B cells from a transgenic non-human mammal whose genome comprises a disruption of an Act1 gene; (B) immortal cells; (C) reagents necessary for loading the B cells with the antigen; (D) reagents necessary for fusing the B cells with an immortal cell; and (E) a package for holding the B cells, the immortal cells, and the reagents.

In some embodiments the kit includes instructions for using the kit to carry out a method of producing a monoclonal antibody specifically directed to an antigen using the B cells, the immortal cells, and the reagents. In certain embodiments, the kit further includes reagents necessary for maintaining the selected hybridoma under conditions in which the monoclonal antibody is expressed. In some embodiments, the mammal is a mouse. In other embodiments, the B cells are present in a spleen cell isolate. In yet other embodiments, the immortal cells of the kit include SP2/0 mouse myeloma cells.

### BRIEF DESCRIPTION OF THE FIGURES

The accompanying drawings, which are incorporated in and constitute a part of this specification, illustrate some embodiments disclosed herein, and together with the description, serve to explain principles of the exemplary embodiments disclosed herein.

FIG. 1 shows a graphical representation of the results of ELISA measurements of supernatants from 38 single hybridoma colonies. Measurement of monoclonal antibody secretion was carried out after 2 weeks of growth at 37° C. in a 10% CO₂ incubator. The ELISA plates were coated with 4 μg/ml delta-giardin. Each column represents the relative antibody levels from individual clonal wells. As shown in Table 1, 14 out of 38 wells had absorbance values >0.40.

### DETAILED DESCRIPTION

The exemplary embodiments disclosed herein will now be described by reference to some more detailed exemplary embodiments. These exemplary embodiments may, however, be embodied in different forms and should not be construed as limited to the embodiments set forth herein. Rather, these exemplary embodiments are provided so that this disclosure will be thorough and complete, and will fully convey the scope of the exemplary embodiments to those skilled in the art.

Unless otherwise defined, all technical and scientific terms used herein have the same meaning as commonly understood by one of ordinary skill in the art to which these exemplary embodiments belong. The terminology used in the description herein is for describing particular exemplary embodiments only and is not intended to be limiting of the exemplary embodiments. As used in the specification and the appended claims, the singular forms “a,” “an,” and “the” are intended to include the plural forms as well, unless the context clearly indicates otherwise. All publications, patent applications, patents, and other references mentioned herein are incorporated by reference in their entirety.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present embodiments. At the very least, and not as an attempt to limit the application of the doctrine of equivalents to the scope of the claims, each numerical parameter should be construed in light of the number of significant digits and ordinary rounding approaches.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

The term antibody, as used herein and unless further limited, refers to single chain, two-chain, and multi-chain proteins and glycoproteins belonging to the classes of polyclonal, monoclonal, chimeric and hetero immunoglobulins; it also includes synthetic and genetically engineered variants of these immunoglobulins. The term “Antibody fragment” includes Fab, Fab′, F(ab′)₂, and Fv fragments, as well as any portion of an antibody having specificity toward a desired target epitope or epitopes.

The term monoclonal antibody, as used herein, refers to an antibody obtained from a population of substantially homogeneous antibodies, i.e., the individual antibodies comprising the population are identical and/or bind the same epitope, except for possible variants that may arise during production of the monoclonal antibody, such variants generally being present in minor amounts. In contrast to polyclonal antibody preparations that typically include different antibodies directed against different epitopes, each monoclonal antibody is directed against a single epitope on the antigen. In addition to their specificity, the monoclonal antibodies are advantageous in that they are uncontaminated by other immunoglobulins. The modifier “monoclonal” indicates the character of the antibody as being obtained from a substantially homogeneous population of antibodies.

The term chimeric antibody, as used herein, refers to an antibody which includes sequences derived from two different antibodies, which typically are of different species. Most typically, chimeric antibodies include human and non-human antibody fragments, generally human constant and non-human variable regions.

The term humanized antibody, as used herein, refers to a type of chimeric antibody derived from a non-human antibody, and a human antibody which retains or substantially retains the antigen-binding properties of the parent antibody but which is less immunogenic in humans.

The term antigen, as used herein, refers to a molecule or a portion of a molecule capable of being bound by an antibody which is additionally capable of inducing an animal to produce an antibody capable of binding to an epitope of that antigen. An antigen can have one or more than one epitope. The specific reaction referred to above is meant to indicate that the antigen will react, in a highly selective manner, with its corresponding antibody and not with the multitude of other antibodies which can be evoked by other antigens.

The term epitope, as used herein, refers to that portion of any molecule capable of being recognized by, and bound by, an antibody. In general, epitopes consist of chemically active surface groupings of molecules, for example, amino acids or sugar side chains, and have specific three-dimensional structural characteristics as well as specific charge characteristics. The epitopes of interest for the present invention are epitopes comprising amino acids.

As described herein, B cells from Act 1 deficient transgenic mice contacted in vitro with an antigen can form IgG antibodies specific to the antigen. As used herein, the term “in vitro” refers to events that occur in an artificial environment, e.g., in a test tube or reaction vessel, in cell culture, in a Petri dish, etc., rather than within an organism (e.g., animal, plant, or microbe). In contrast, the term “in vivo”, as used herein, refers to events that occur within an organism (e.g., animal, plant, or microbe or cell or tissue thereof).

One aspect of this invention presents a method of producing IgG type monoclonal antibodies. The method includes the steps of: (A) providing B-cells obtained from a transgenic non-human mammal whose genome comprises a disruption of an Act1 gene; (B) contacting the B cells with an antigen under conditions which result in formation of an antibody specific to the antigen by the B cells; (C) fusing the B cells with one or more immortal cells, to produce one or more hybridomas which express IgG type monoclonal antibodies that specifically bind to the antigen; (D) selecting a hybridoma which expresses an antibody specific to the antigen; and (E) obtaining monoclonal antibody produced by the selected hybridoma.

In accordance with the present invention, B cells provided for use in a method of the present invention can be obtained from a transgenic non-human mammal whose genome comprises a disruption of an Act1 gene. As used herein, the term “B cell” refers to lymphocytes that originate in the bone marrow and produces antibodies. B cells play a role in the humoral immune response and are a component of the adaptive immune system. B cells are usually isolated from the spleen, tonsils, bone marrow or peripheral blood of mammals. In this application the expressions “B cell”, “B-cell” and “B lymphocyte” refer to the same cell.

The present invention provides IgG monoclonal antibodies and antibody fragments specific for a desired antigen. As used herein, the term IgG refers to a polypeptide belonging to the class of antibodies that are substantially encoded by a recognized immunoglobulin gamma gene. The IgG polypeptide comprises four peptide chains, two identical heavy chains and two identical light chains arranged in a Y-shape typical of antibody monomers. In humans this class comprises IgG1, IgG2, IgG3, and IgG4. In mice this class comprises IgG1, IgG2a, IgG2b, and IgG3.

Antibodies are designed for specific binding, as a result of the affinity of complementary determining region of the antibody for the epitope of the target molecule. For example, an antibody specific for a chosen antigen can be an antibody or antibody fragment capable of binding to that specific protein with a specific affinity of between 10⁻⁸ M and 10⁻¹¹ M. In some embodiments, an antibody or antibody fragment binds to a selected antigen with a specific affinity of greater than 10⁻⁷M, 10⁻⁸M, 10⁻⁹M, 10⁻¹⁰M, or 10⁻¹¹M, between 10⁻⁸M-10⁻¹¹M, 10⁻⁹M-10⁻¹⁰M, and 10⁻¹⁰M-10⁻¹¹M. In a preferred aspect, specific activity is measured using a competitive binding assay as set forth in Ausubel FM, (1994). Current Protocols in Molecular Biology. Chichester: John Wiley and Sons (“Ausubel”), which is incorporated herein by reference.

### Act1 Knockout Mammals

A transgenic non-human mammal which lacks a functional Act1 gene is referred to herein as a “transgenic non-human Act1 knockout mammal” or an “Act1 knockout mammal”. In a particular embodiment, the genome of the Act1 knockout mammal comprises at least one non-functional allele for the endogenous Act1 gene.

Mice being utilized as the non-human mammal whose genome comprises a disruption of an Act1 gene of choice for the purposes of example only. The breadth of the invention is not limited to the utilization of mice, indeed any suitable non-human mammal in which the methods described herein could be applied by those skilled in the art is encompassed by the invention. For example, a suitable mammal can be a rodent, a canine, a feline, an ovine, a bovine, a porcine and a caprine. Specific examples of suitable mammals include a mouse, a rat, a dog, a cat, a sheep, a cow, a pig, a goat and a rabbit.

As used herein, the term “gene” refers to DNA sequences which encode the genetic information (for example, nucleic acid sequence) required for the synthesis of a single protein (for example, polypeptide chain). The term “Act1 gene” refers to a particular mammalian gene which comprises a DNA sequence which encodes the Act1 protein. Chang et al., J. Biol. Chem. 281, 35603-35607 (2006). An “allele” is an alternative from of gene found at the same locus of a homologous chromosome. Homologous chromosomes are chromosomes which pair during meiosis and contain identical loci. The term locus connotes the site (for example, location) of a gene on a chromosome.

As used herein the terms “transgenic non-human Act1 knockout mammal” and “Act1 knockout mammal” refer to a mammal whose genome comprises a disrupted or inactivated Act1 gene. Those of skill in the art will recognize that the term “knockout” refers to the functional inactivation (knockdown) of the gene. The disruption introduces a chromosomal defect (for example, mutation or alteration) in the Act1 gene at a point in the nucleic acid sequence that is important to either the expression of the Act1 gene or the production of a functional Act1 protein (for example, polypeptide). The disruption can also introduce a chromosomal defect in a region other than the Act1 gene wherein the disruption results in an inactivated Act1 gene. Thus, the introduction of the disruption inactivates the endogenous target gene (for example, Act1 gene).

Functional inactivation of the Act1 gene can include the partial or complete reduction in the expression and/or function of the Act1 polypeptide encoded by the endogenous gene of a single type of cell, selected cells (for example, B cells) or all of the cells of a non-human transgenic Act1 knockout animal. Thus, according to the instant invention the expression or function of the Act1 gene product can be completely or partially disrupted or reduced (for example, by about 50%, 75%, 80%, 90%, 95% or more) in a selected group of cells (for example, a tissue or organ) or in the entire animal. As used herein, the term “a functionally disrupted Act1 gene” includes a modified genome wherein the modification in the genome results in failure of expression of Act1 polypeptide (partially such as low levels of expression, completely such as lack of expression) or expression of a nonfunctional (partially, completely) Act1 protein; and a modified Act1 gene which fails to express an Act1 polypeptide or which expresses an Act1 polypeptide that lacks completely or partially the biologically activity of Act1 (e.g., a truncated polypeptide having less than the entire amino acid polypeptide chain of a wild-type Act1 polypeptide and is partially or completely non-functional; a mutated Act1 polypeptide which is partially or completely non-functional).

In a particular embodiment, the invention provides a transgenic knockout mammal whose genome comprises either a homozygous or heterozygous disruption of its Act1 gene. A knockout mammal whose genome comprises a homozygous disruption is characterized by somatic and germ cells which contain two nonfunctional (disrupted) alleles of the Act1 gene while a knockout mutant whose genome comprises a heterologous disruption is characterized by somatic and germ cells which contain one wild-type allele and one nonfunctional allele of the Act1 gene.

As used herein, the term “genotype” refers to the genetic makeup of an animal with respect to the Act1 chromosomal locus. More specifically the term genotype refers to the status of the animal's Act1 alleles, which can either be intact (for example, wild-type or +/+); or disrupted (for example, knockout) in a manner which confers either a heterozygous (for example, +/−); or homozygous (−/−) knockout genotype.

In exemplary embodiments, the transgenic mammal whose genome comprises a disruption of an Act1 gene non-human mammal is an Act1 knockout mouse. Act-1 deficient mice can be prepared as described in (Qian et al., Immunit, 21:575-587 (2004)). Briefly, the standard methodology for producing a transgenic embryo requires introducing a targeting construct, which is designed to integrate by homologous recombination with the endogenous nucleic acid sequence of the targeted gene, into a suitable embryonic stem (ES) cells. The ES cells are then cultured under conditions effective for homologous recombination between the recombinant nucleic acid sequence of the targeting construct and the genomic nucleic acid sequence of the host cell chromosome. Genetically engineered stem cells that are identified as comprising a knockout genotype which comprises the recombinant allele is introduced into an animal, or ancestor thereof, at an embryonic stage using standard techniques which are well known in the art (for example, by microinjecting the genetically engineered ES cell into a blastocyst). The resulting chimeric blastocyst is then placed within the uterus of a pseudo-pregnant foster mother for development into viable pups. The resulting viable pups include potentially chimeric founder animals whose somatic and germline tissue comprise a mixture of cells derived from the genetically-engineered ES cells and the recipient blastocyst. The contribution of the genetically altered stem cell to the germline of the resulting chimeric mice allows the altered ES cell genome which comprises the disrupted target gene to be transmitted to the progeny of these founder animals thereby facilitating the production of transgenic “knockout animals” whose genomes comprise a gene which has been genetically engineered to comprise a particular defect in a target gene.

According to techniques well known to those of skill in the art, genetically engineered (for example, transfected using electroporation or transformed by infection) embryonic stem (ES) cells, are routinely employed for the production of transgenic non-human embryos. ES cells are pluripotent cells isolated from the inner cell mass of mammalian blastocyst. ES cells can be cultured in vitro under appropriate culture conditions in an undifferentiated state and retain the ability to resume normal in vivo development as a result of being combined with blastocyst and introduced into the uterus of a pseudo-pregnant foster mother. Those of skill in the art will recognize that various stem cells are known in the art, for example AB-1, HM-1, D3. CC1.2, E-14T62a, RW4 or JI (Teratomacarcinoma and Embryonic Stem Cells: A Practical Approach, E. J. Roberston, ed., IRL Press).

It is to be understood that the Act1 knockout mammals described herein can be produced by methods other than the ES cell method described above, for example by the pronuclear injection of recombinant genes into the pronuclei of one-cell embryos or other gene targeting methods which do not rely on the use of a transfected ES cell, and that the exemplification of the single method outlined above is not intended to limit the scope of the invention to animals produced solely by this protocol.

The transgenic Act1 knockout mammals described herein can also be bred (for example, inbred, outbred or crossbred) with appropriate mates to produce colonies of animals whose genomes comprise at least one non-functional allele of the endogenous gene which naturally encodes and expresses functional Act1. Examples of such breeding strategies include but are not limited to: crossing of heterozygous knockout animals to produce homozygous animals; outbreeding of founder animals (for example, heterozygous or homozygous knockouts), or with a non-human mammal, such as a mouse, whose inbred genetic background has been altered.

As a result of the disruption of the Act1 gene, the Act1 knockout mammal of the present invention can manifest a particular phenotype. The term phenotype refers to the resulting biochemical or physiological consequences attributed to a particular genotype. In one embodiment, the Act1 knockout mammal displays lymphoid system abnormalities (e.g., enlarged lymph nodes (e.g., cervical, axillary, brachial), lymphoid hyperplasia, increased germinal centers, accumulation of large numbers of immunoglobulin-producing plasma cells (Syndecan-1 positive cells) in the medulla of the lymph nodes); hypergammaglobulinemia; production of autoantibodies and combinations thereof. The phenotype can further comprise inflammation of tissue (e.g., upper respiratory airway, skin). The Act1 knockout mammal can also display SLE and Sjögren's syndrome. In addition, the phenotype of the Act1 knockout non-human cell or mammal can include the development of cancer (e.g., lung adenoma, skin fibroepithelioma).

### Methods of Disrupting the Act1 Gene

Disruption of the Act1 gene can be accomplished by a variety of methods known to those of skill in the art. For example, gene targeting using homologous recombination, mutagenesis (for example, point mutation), RNA interference (e.g., small interfering RNA (siRNA), short hairpin RNA (shRNA)) and anti-sense technology can be used to disrupt an Act1 gene.

In a particular embodiment of the present invention, a transgenic Act1 knockout mammal is produced by introducing a targeting vector which disrupts the Act1 gene into an ES cell thereby producing a transgenic stem cell. A transgenic ES cell which includes the disrupted Act1 gene due to the integration of the targeting vector into its genome is selected and introduced into a blastocyst, thereby forming a chimeric blastocyst. The chimeric blastocyst is introduced into the uterus of a pseudo-pregnant mammal wherein the pseudo-pregnant mammal gives birth to a transgenic non-human mammal which lacks a functional Act1 gene.

One of skill in the art will easily recognize that the Act1 gene can be disrupted in a number of different ways, any one of which may be used to produce the Act1 knockout mammals of the present invention. For example, a transgenic knockout animal according to the instant invention can be produced by the method of gene targeting. As used herein the term “gene targeting” refers to a type of homologous recombination which occurs as a consequence of the introduction of a targeting construct (for example, vector) into a mammalian cell (for example, an ES cell) which is designed to locate and recombine with a corresponding portion of the nucleic acid sequence of the genomic locus targeted for alteration (for example, disruption) thereby introducing an exogenous recombinant nucleic acid sequence capable of conferring a planned alteration to the endogenous gene. Thus, homologous recombination is a process (for example, method) by which a particular DNA sequence can by replaced by an exogenous genetically engineered sequence. More specifically, regions of the targeting vector which have been genetically engineered to be homologous (for example, complementary) to the endogenous nucleotide sequence of the gene which is targeted for disruption line up or recombine with each other such that the nucleotide sequence of the targeting vector is incorporated into (for example, integrates with) the corresponding position of the endogenous gene.

One embodiment of the present invention provides a vector construct (for example, an Act1 targeting vector or Act1 targeting construct) designed to disrupt the function of a wild-type (endogenous) mammalian Act1 gene. In a particular embodiment, an effective Act1 targeting vector comprises a recombinant sequence that is effective for homologous recombination with the Act1 gene. For example, a replacement targeting vector comprising a genomic nucleotide sequence which is homologous to the target sequence operably linked to a second nucleotide sequence which encodes a selectable marker gene exemplifies an effective targeting vector. Integration of the targeting sequence into the chromosomal DNA of the host cell (for example, an ES cell) as a result of homologous recombination introduces an intentional disruption, defect or alteration (for example, insertion, deletion) into the sequence of the endogenous gene. One aspect of the present invention is to delete, replace (e.g., mutate) all or part of the nucleotide sequence of a non-human mammalian gene which encodes the Act1 polypeptide. In one embodiment, an (one or more) exon(s) of the Act1 gene is disrupted (e.g., exon 1, exon 2, exon 3, exon 4, exon 5, exon 6, exon 7, exon 8, exon 9). In another embodiment, a portion of an exon (or portions of one or more exons) is disrupted. In a particular embodiment, a segment which includes exon 2 of the Act1 gene, is removed.

One of skill in the art will recognize that any Act1 genomic nucleotide sequence of appropriate length and composition to facilitate homologous recombination at a specific site that has been preselected for disruption can be employed to construct a Act1 targeting vector. Guidelines for the selection and use of sequences are described for example in Deng and Cappecchi, Mol. Cell. Biol., 12:3365-3371 (1992) and Bollag, et al., Annu. Rev. Genet., 23:199-225 (1989). For example, a wild-type Act1 gene can be mutated and/or disrupted by inserting a recombinant nucleic acid sequence (for example, a Act1 targeting construct or vector) into all or a portion of the Act1 gene locus. For example, a targeting construct can be designed to recombine with a particular portion within the enhancer, promoter, coding region, start codon, non-coding sequence, introns or exons of the Act1 gene. Alternatively, a targeting construct can comprise a recombinant nucleic acid which is designed to introduce a stop codon after one or more exons of the Act1 gene. In a particular embodiment, an Act1 gene targeting construct comprises a 5′ arm comprising the first intron of the Act1 gene, followed by a first recombination site (e.g., loxP site), followed by a marker gene (e.g., Neo gene), followed by a recombination site, followed by one or more exons of the Act1 gene (e.g., exon 2), followed by a recombination site, followed by a 3′ arm comprising a fragment from the second intron of the Act1 gene.

Suitable targeting constructs of the invention can be prepared using standard molecular biology techniques known to those of skill in the art. For example, techniques useful for the preparation of suitable vectors are described by Maniatis, et al., Molecular Cloning: A Laboratory Manual, Cold Spring Harbor Laboratory, Cold Spring Harbor, N.Y. Appropriate vectors include a replacement vector such as the insertion vector described by Capecchi, M. R., Science, 244:1288-92 (1989); or a vector based on a promoter trap strategy or a polyadenylation trap, or “tag-and-exchange” strategy as described by Bradley, et al., Biotechnology, 10:543-539 (1992); and Askew, et al., Mol. Cell. Biol., 13:4115-5124 (1993).

One of skill in the art will readily recognize that a large number of appropriate vectors known in the art can be used as the basis of a suitable targeting vector. In practice, any vector that is capable of accommodating the recombinant nucleic acid sequence required to direct homologous recombination and to disrupt the target gene can be used. For example, pBR322, pACY164, pKK223-3, pUC8, pKG, pUC19, pLG339, pR290, pKC11 or other plasmid vectors can be used. Alternatively, a viral vector such as the lambda gt11 vector system can provide the backbone (for example, cassette) for the targeting construct.

### Preparation of Immortalized Cell Line

Transgenic Act1 knockout mammals produced in accordance with the present invention are utilized as a source of immune cells for the establishment of immortalized cell lines, which are useful for the production of IgG monoclonal antibodies in accordance with the present invention. Such cells, which can be isolated from Act1 knockout mammalian tissues, include lymphocytes capable of producing antibodies, for example, B cells.

In some embodiments, B cells provided for use in the present invention can be obtained from lymphoid organs that have been surgically removed from one or more Act1 knockout mammals. Lymphoid organs from which B cells can be obtained include the spleen, thymus or lymph nodes of Act1 knockout mammals. In particular embodiments, the B cells are present in a spleen cell isolate obtained from an Act1 knockout mammal. By way of example, single cell suspensions of B cells can be obtained from a spleen using well known methods of mechanical tissue separation (e.g., through the use of a cell strainer, tissue separation sieve or similar device). In some embodiments, it is preferable to retain the B cells as part of a lymphoid cell isolate (e.g., a spleen cell isolate) which includes other cells and factors found in the lymphoid tissue.

B cells obtained from transgenic Act1 knockout mammals for use in the present invention can be fresh or stored (e.g., frozen). Frozen B cells can be stored for varying amounts of time, such as being stored for an hour, a day, a week, a month, or more than a month. In an exemplary embodiment, B cells are isolated from the spleens of Act1 knockout mice and frozen at −70° C. in a freezing media including 10% dimethyl sulfoxide (DMSO) and 90% Fetal Bovine Serum (FBS). Once frozen, the B cells can then be thawed and provided as needed for use in the present invention.

In some embodiments, an effective amount of a commercially available hybridoma cloning supplement (HCS) can be added to a fusion reaction media or directly to B cells suspended in a cell culture media. As a culture media supplement, HCS includes growth factors which can promote the growth and development of hybridoma cells in a manner similar to the use of feeder cells without the disadvantages of feeder cells such as: overgrowth of newly formed hybridomas; source of contamination; competition for nutrients; and variations in growth factor concentration For example, a fusion reaction media can include 5% v/v of HCS. In some embodiments, an effective amount of anti-CD40 monoclonal antibodies can be added to a fusion reaction media or directly to B cells already suspended in a cell culture media in order to improve B cell viability during antigen exposure in culture. In certain embodiments, about 1 to about 2 μg/ml of anti-CD40 monoclonal antibody (BD Pharmingen) can be included in a suitable growth media during antigen exposure. Additional agents for use in a fusion reaction media of the present invention can include an effective amount of IL-6, GM-CSF and/or GSF.

Contacting B cells with Antigen

In accordance with the method, B cells provided as described herein are contacted with an antigen under conditions which result in formation of an antibody specific to the antigen by the B cells. As used herein the term contacting refers to bringing about direct contact between the B cell and the antigen such that the B cell and the antigen are in immediate proximity or association with each other. An antigen according to the present invention may be selected from (human) pathogens, allergens, bacteria, toxins (such as endotoxin, enterotoxins, etc.), mycoplasma, fungi and viruses as well as any parts of such antigens. In some embodiments, the antigen may be a peptide, pure protein, partially purified protein or perhaps a non-purified tissue sample. It may also be soluble or insoluble.

In some embodiments, the antigen can include a toxic and/or pyrogenic antigen that may kill or sicken an animal during the animal immunization step of a standard monoclonal antibody production method. Many pathogens, whether they are bacteria, viruses, mycoplasma or other disease-causing toxins, agents or substances, have a direct effect on the survivability of the affected host. Therefore, a direct immunization is not possible on account of the high toxicity of many pathogenic substances. Because the present invention produces monoclonal antibodies from cell culture, antigens that would otherwise be too toxic or pyrogenic for in vivo administration, but remain suitable for in vitro administration, can still be used.

B cells provided in a suitable cell culture media can be contacted with an antigen by adding (e.g., pipetting) the antigen directly to B cells suspended in the media. In other embodiments, B cells can be re-suspended in a fusion reaction media, the fusion reaction media including a suitable cell culture media and an effective amount of antigen.

The amount and concentration of antigen administered to the B cells is the amount and at a concentration sufficient to stimulate the B cells to form antibodies specific to the antigen. The amount and concentration of the antigen can vary depending on a variety of factors, such as the immunogenicity of the antigen. In some embodiments, the amount of antigen administered to the B cells is substantially less than what would be required to stimulate antibody formation in a conventional monoclonal antibody preparation system. For example, in some embodiments, the amount of antigen administered can be about 50% less, about 75% less, or about 90% less than that typically administered to a live animal immunized as part of a monoclonal antibody production protocol.

In some embodiments, B cells are exposed to an antigen for about 7 to about 21 days. In particular embodiments, B cells are exposed to an antigen for about 10 to about 14 days. During exposure of B cells to an antigen, culture media can be removed and replaced with an equal amount of fresh media (e.g., the reaction media) as needed. For example, half the volume of media can be replaced at Day 4 and/or all of the media can be replaced with fresh media at Day 7. In exemplary embodiments, the viability of the B cells remains >50% after 10 days in tissue culture in the presence of the antigen.

### Preparation of Hybridoma Cells

Following contact with an antigen, B cells are fused with one or more immortal cells to produce one or more hybridomas which express IgG type monoclonal antibodies that specifically bind to the antigen. As used herein, the term “immortal cells” refers to a cell line that is immortalized, e.g., under suitable in vitro conditions, the cell line divides virtually indefinitely. In some embodiments, immortal cells are capable of growing in culture for more than 35 passages. In some embodiments, the fusion is catalyzed by the addition of a glycoprotein fusogen, e.g., polyethylene glycol (PEG) to a suspension of B cells and immortal cells that facilitates the fusion of cell to cell membranes. The success of the procedure is very much dependent on the skill and experience of the operator, as the freshly fused cells are sensitive to mechanical and chemical disruption.

Therefore, the step of fusing the B cells with an immortal cell can include the use of a polyethylene glycol (PEG) polymer complex. In an exemplary embodiment, the PEG polymer complex includes 0.5 g/ml PEG 4000+10% DMSO. In some embodiments, the immortal cell includes a mouse myeloma cell. In certain embodiments, the mouse myeloma cell includes a SP2/0 mouse myeloma cell. In an exemplary embodiment, SP2/0 mouse myelomas and spleen derived B cells are suspended at a ratio of 1:4 in 10 ml DMEM+pyruvate+2% FBS and fused using a standard PEG fusion protocol.

PEG is used to fuse adjacent plasma membranes, but the success rate is low so a selective medium in which only fused cells can grow is used. This is possible because myeloma cells have lost the ability to synthesize hypoxanthine-guanine-phosphoribosyl transferase (HGPRT), an enzyme necessary for the salvage synthesis of nucleic acids. The absence of HGPRT is not a problem for these cells unless the de novo purine synthesis pathway is also disrupted. By exposing cells to aminopterin (a folic acid analogue, which inhibits dihydrofolate reductase, DHFR), they are unable to use the de novo pathway and become fully auxotrophic for nucleic acids requiring supplementation to survive.

Successfully fused cells (hybridomas) are selected using HAT medium. The selective culture medium is called HAT medium because it contains hypoxanthine, aminopterin, and thymidine. This medium is selective for fused (hybridoma) cells. Unfused myeloma cells cannot grow because they lack HGPRT, and thus cannot replicate their DNA. Unfused B cells cannot grow indefinitely because of their limited life span. Only fused hybrid cells, referred to as hybridomas, are able to grow indefinitely in the media because the B cell partner supplies HGPRT and the myeloma partner has traits that make it immortal (similar to a cancer cell). Removal of the unfused myeloma cells is necessary because they have the potential to outgrow other cells, especially weakly established hybridomas.

This mixture of cells is then diluted and clones are grown from single parent cells on microtitre wells.

The success of the fusion between the antigen exposed B cells and immortal cells, such as a myeloma cell line, may be controlled—at least to some extent—by a number of factors. These include, although not exclusively; the fusogen, temperature, cell mixing protocol, ratio of spleen to myeloma cells, time of fusion, cell fusion recovery protocol, media/buffer batches and of course the investigator. The precise details of the protocol and success of the fusion often vary from laboratory to laboratory and even from experiment to experiment. An alternative method to the use of PEG as the fusogen may include ‘electrofusion’, where an electrical field is used to fuse the B cells with myelomas.

Selecting a Hybridoma which Expresses Antibody Specific to the Antigen

Following fusion between one or more of the antigen exposed B cells and immortal cells described herein, a hybridoma which expresses an antibody specific to the antigen is selected.

Hybridomas which express antibodies (e.g., IgG antibodies) that specifically bind to the antigen can be identified and selected using a variety of methods well known in the art. In some embodiments, the step of selecting a hybridoma includes the use of an immunoassay or other functional screening assays. In some embodiments, the immunoassay is an ELISA assay. By way of example, the antibodies secreted by the different hybridoma clones can be assayed for their ability to bind to the antigen of interest (i.e., the selected antigen contacted with B cells in accordance with the present invention described above) using an ELISA assay. Alternatively, more sophisticated assays that closely mimic the final application anticipated for the antibody may be used such as scintillation-proximity, fluorescence or fluorescence polarization assays.

The term “ELISA” includes an enzyme-linked immunosorbent assay that employs an antibody or antigen bound to a solid phase and an enzyme-antigen or enzyme-antibody conjugate to detect and quantify the amount of an antigen or antibody present in a sample. A description of the ELISA technique is found in Chapter 22 of the 4th Edition of Basic and Clinical Immunology by D. P. Sites et al., 1982, published by Lange Medical Publications of Los Altos, Calif. and in U.S. Pat. Nos. 3,654,090; 3,850,752; and 4,016,043, the disclosures of which are herein incorporated by reference. ELISA is an assay that can be used to quantitate the amount of antigen specific monoclonal antibody in a sample obtained from a given hybridoma supernatant. In present embodiments, an ELISA can be carried out by attaching on a solid support (e.g., polyvinylchloride) an antigen of interest. Supernatant including antibodies secreted by the different clones are be added for formation of an antibody-antigen complex, and the extra, unbound sample is washed away. An enzyme-linked antibody, specific for the antigen-specific IgG antibodies (e.g., an anti-mouse IgG antibody) is then added. The support is washed to remove the unbound enzyme-linked second antibody. The enzyme-linked antibody can include, but is not limited to, alkaline phosphatase. The enzyme on the second antibody can convert an added colorless substrate into a colored product or can convert a non-fluorescent substrate into a fluorescent product. The ELISA-based assay method provided herein can be conducted in a single chamber or on an array of chambers and can be adapted for automated processes.

A selected hybridoma which expresses an antibody specific to the antigen can be maintained under conditions in which the monoclonal antibody is stably expressed. A selected hybridoma can then be cloned. Hybridomas are expanded in cell culture to produce monoclonal antibody. Monoclonal antibodies produced by the selected hybridoma can be obtained using a variety of well known methods.

The cultivation of individual IgG monoclonal antibody secreting hybridoma clones is conducted using generally recognized cell cultivation procedures as well as optimized procedures used for commercial and industrial applications. The step of obtaining monoclonal antibodies occurs using the current state of the art as well as optimized laboratory techniques. By way of example, once identified and selected, hybridoma clones capable of producing IgG monoclonal antibodies that specifically bind to the antigen of interest can then be sub-cultured from, for example, the micro-titer plate into six well plates then into T75 flasks. Further subculturing either in flasks or at bioreactor scale may then be performed in order to yield sufficient antibody as required.

### Purification and Modification of Antibodies

Purification of the IgG monoclonal antibodies produced in accordance with the present invention can be accomplished by a variety of methods known to those of skill including, precipitation by ammonium sulfate or sodium sulfate followed by dialysis against saline, ion exchange chromatography, affinity or immunoaffinity chromatography as well as gel filtration, zone electrophoresis, etc. (Godingin, Monoclonal Antibodies: Principles and Practice, 2d ed., pp. 104-126, Orlando, Fla., Academic Press). It is preferable to use purified antibodies or purified fragments of the antibodies having at least a portion of an antigen binding region, including such as Fv, F(ab′)₂, Fab fragments (Harlow and Lane, 1988, Antibody, Cold Spring Harbor Laboratory Press) for the detection of the antigen in a subsequent method.

Antibodies specifically reactive with the antigen produced in accordance with the present invention may be used in any known immunoassays which rely on the binding interaction between an antigenic determinant of a protein and the antibodies. Examples of such assays are radioimmunoassays, enzyme immunoassay (e.g., ELISA), immunofluorescence, immunoprecipitation, latex agglutination, hemagglutination, and histochemical tests.

In some embodiments, the monoclonal antibodies obtained are chimeric antibodies such as humanized monoclonal antibodies. The successful application of mouse derived monoclonal antibodies for human therapeutic applications has been hindered due to the immunogenicity in man of the mouse antibody, independent of its antigen specificity. Such human anti-mouse antibodies (HAMA) neutralize the administered antibody and can also lead to toxicity in the patient. Thus, there is great potential for the production of humanized monoclonal antibodies, specifically for therapeutic applications. Humanizing IgG monoclonal antibodies produced in accordance with the present invention can be accomplished by a variety of methods known to those of skill in the art.

By way of example, the production of humanized antibodies may be achieved by crossing the transgenic Act1 knockout mice described herein with a transgenic line where the mouse Ig genes have been replaced with their human equivalent. Such a line is available in the form of the ‘Xenomouse’ developed by Abgenix and described in U.S. Pat. No. 6,114,598. The ‘Xenomouse’ is presented by way of example as an established method. An extension of the Xenomouse technology is described in U.S. Pat. No. 6,207,418. GenPharm international Inc. (now Medarex) developed a similar mouse technology (HuMab) which can be used for generating humanized monoclonal antibodies, see U.S. Pat. No. 5,877,397and U.S. Pat. No. 5,874,299.

### Kits for Producing IgG Monoclonal Antibodies

In another aspect, the invention provides kits that include the elements for producing an IgG monoclonal antibody specifically directed to an antigen in vitro. For example, kits can include B cells from a transgenic non-human mammal whose genome comprises a disruption of an Act1 gene. Kits may also include: immortal cells; reagents necessary for loading the B cells with the antigen; reagents necessary for fusing the B cells with an immortal cell; and a package or container for holding the B cells, the immortal cells, and the reagents. The reagents included in the kit will vary depending on the particular technique being used to select hybridomas producing antigen-specific monoclonal antibodies.

In certain embodiments, the kit includes B cells from the transgenic non-human mammal whose genome comprises a disruption of an Act1 gene is a mouse, (e.g., an Act1 knockout mouse). The B cells can include B cells present in a spleen cell isolate obtained from an Act1 knockout mouse.

Immortal cells in a kit of the present invention can include mouse myeloma cells. In certain embodiments, the mouse myeloma cells are SP/0 mouse myeloma cells.

The reagents may be supplied in a solid (e.g., lyophilized) or liquid form. The kits of the present invention may optionally comprise different containers (e.g., vial, ampoule, test tube, flask or bottle) for each individual buffer, cell type and/or reagent. Each component will generally be suitable as aliquoted in its respective container or provided in a concentrated form. Other containers suitable for conducting certain steps of the disclosed methods may also be provided. The individual containers of the kit are preferably maintained in close confinement for commercial sale.

For example, the kit can include a carrier for the various components of the kit. The carrier can be a container or support in the form of, e.g., a bag, box, tube or rack, and is optionally compartmentalized. The carrier may define an enclosed container for safety purposes during shipment and storage.

In some embodiments, the kit further comprises instructions for using the kit to carry out a method of producing a monoclonal antibody specifically directed to an antigen using the B cells, the immortal cells, and the necessary reagents. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

In some embodiments, the kit further comprises reagents necessary for maintaining the selected hybridoma under conditions in which the IgG monoclonal antibody specific to the antigen of interest is expressed. The kit can also include a solid support such as microtiter multi-well plates, standards, assay diluent, wash buffer, and adhesive plate covers.

In an exemplary embodiment, a kit can be manufactured that includes frozen Act1 knockout mouse spleen derived B cells, SP2/0 myeloma cells, PEG/DMSO, and other standard tissue culture mediums described herein. Specific instructions describing this method in detail are included.

The following example is included for purposes of illustration and is not intended to limit the scope of the invention.

### EXAMPLE

Current in vitro methods using normal mouse spleens produce predominantly IgM monoclonal antibodies. However, the preferred isotype for monoclonal antibodies is the IgG isotype monoclonal antibody. We have developed an in vitro method for the exclusive production of IgG monoclonal antibodies.

### Experimental Procedures

### Isolation of Spleen Cells

Act1 knockout mice were sacrificed by cervical dislocation and the skin sterilized with ethanol. All further manipulations were performed under sterile conditions. The spleen was removed and placed into a 100×15 mm dish containing DMEM-high glucose+pyruvate+2% FBS. The dish containing the spleen was transferred to a cell culture hood where the spleen was transferred to a new dish with fresh media and the old dish and media were discarded.

The spleen was then cleaned of extraneous tissue or fascia on the surface of the organ. Fascia that is not removed can be a source of fibroblasts which will quickly over grow any of the fused cells in a well. After cleaning the spleen was transferred to a new dish and a minimal amount of media (just enough to cover the organ) was added to the dish. Using a sterile scalpel and blade the spleen was minced until the capsule of the organ was broken down and the spleen is in pieces.

The media containing the minced spleen was then transferred to a cellector tissue sieve in a 100×15 mm dish where enough media to cover the screen was added. The tissue was then pushed through the 40 mesh screen using a pestle. The process was completed when white or opaque tissue remains on top of the screen.

The entire cell suspension was then transferred to a sterile 50 ml Bluemax Tube (Becton Dickinson) and centrifuged at 270×g for 5 minutes. After centrifugation the supernatant was removed and the pellet was re-suspended in 10 ml of DMEM-high glucose+pyruvate+2% FBS media. The re-suspended pellet was collected into a 10 ml pipette and held horizontally while slowly dripping the suspension down the side of the 50 ml tube. This removes the majority of the connective tissue and clumps of tissue that would interfere with the fusion process. The cells were then counted. Typical spleens resulted in 2-3×10⁸ cells.

The suspension was re-centrifuged at 270×g for 5 minutes and the supernatant removed. The pellet was re-suspended in 2 ml of freezing media (90% FBS, 10% DMSO). The re-suspended spleen cells were then split into cryovials and frozen at 1×10⁸ cells/vial. A typical spleen resulted in 2-3 frozen vials.

### Antigen Exposure

3 vials of Act1 KO spleen cells (6-7×10⁸ cells total) described above were thawed out and centrifuged at 270×g for 5 minutes. The supernatant was removed and the pellet resuspended in 24 ml DMEM+pyruvate with 5%FBS, 5% HCS (hybridoma cloning supplement, PAA Cat #3F05-009), 1 μg/mlanti-CD40 MoAb, 2 μg/ml delta-giardin antigen. 12 ml of the resuspended mixture was pipette into 2 10 mm petri dishes. Each petri dish was placed in a 15 cm petri dish with milli-Q water (Millipore) in order to prevent evaporation.

On Day 4 we removed half (˜6 ml) of reaction media, centrifuged the media at 270×g and removed the supernatant. We then replaced the supernatant with equal volume of fresh media. On Day 7 we removed and replaced all the media.

### Cell Fusion

On day 10 of antigen exposure, all the Act1 KO spleen cells were removed from the petri dishes and counted. At this point, we observed that the cells were >50% viable after 10 days of antigen exposure. The cells were centrifuged at 270×g and re-suspended in 10 ml DMEM+pyruvate+2% FBS and then counted again. SP2/0 mouse myeloma cells totaling ¼ of the number of spleen cells were prepared in suspension. A suspension of myelomas and spleen cells were then prepared at a ratio of 1:4 (myelomas:spleen cells) and centrifuged at 270×g for 5 minutes. The supernatant was then aspirated off of the mixed pellet and the tube was then tapped to loosen the remaining pellet.

1 ml of PEG solution (PEG 4000, 2 g/4 ml, +10% DMSO) was added to the mixed myeloma/spleen pellet over a one minute period while rotating the tube. While continuing to rotate the tube, the tube was incubated in a 37° water bath for one minute. While still rotating the tube, 1.0 ml of DMEM-high glucose+pyruvate was added to the tube over one minutes time and then 15 ml of DMEM-high glucose+pyruvate was added to the tube over a 3 minute period. After all the media was been added, the suspension was centrifuged at 270×g for 5 minutes and the supernatant removed. The final pellet was suspended in DMEM-high glucose+pyruvate+10% FBS+1× methotrexate (ME)+1 hypoxanthine-thymidine (HT)+10% HCS at a concentration of 8×10⁵ original myeloma cells per ml of media.

100 μl of the fused cell suspension was then added to the inner 60 wells of 96 well plates and incubated for 16-24 hours at 37° C. with 10% CO₂ and humidity. After 16-24 hours, 100 μl of DMEM-high glucose+pyruvate+10% FBS+1× methotrexate (ME)+1× hypoxanthine-thymidine (HT)+2× Aminopterin+10% HCS was added to all wells. After about 5 days post fusion the initial signs of viable hybridoma colonies were observable in the wells. By 12-14 days post fusion the hybridoma colonies were macroscopically visible and ready for the screening assay. Two successful fusions of the spleen cells were performed with viable hybridomas (>90% of wells).

### Screening Assays

Hybridomas producing antigen specific antibodies to delta-giardin antigen were selected by ELISA and cloned. 100% IgG production was confirmed experimentally (>400 wells) by ELISA. Delta-giardin antigen-specific antibodies were detected by ELISA in the first screening of hybridomas. Stable clones (producing specific antibody) were then isolated and frozen back.

Cells from well number 4B2 in the original fusion screening were re-suspended and cloned by limiting dilution in DMEM, 10% FBS, 10% HCS (hybridoma cloning supplement, PAA cat. 3 F05-009). After 2 weeks of growth at 37° C. in a 10% CO₂ incubator, 38 single hybridoma colonies were visible (from 120 total wells plated). Supernatants were removed and tested for the presence of antibody by ELISA using ELISA plates coated with 4 μg/ml delta-giardin antigen. As shown in FIG. 2, each column represents the relative antibody levels from individual clonal wells. As shown in Table 1 below, 14 out of 38 wells had OD490 absorbance values >0.40.

TABLE 1

| Well number | OD490 |
|---|---|
| 1 | 0.09 |
| 2 | 0.50 |
| 3 | 0.11 |
| 4 | 0.09 |
| 5 | 0.59 |
| 6 | 0.70 |
| 7 | 0.13 |
| 8 | 0.10 |
| 9 | 0.09 |
| 10 | 0.11 |
| 11 | 0.08 |
| 12 | 0.91 |
| 13 | 0.75 |
| 14 | 0.16 |
| 15 | 0.22 |
| 16 | 0.11 |
| 17 | 0.09 |
| 18 | 0.81 |
| 19 | 0.22 |
| 20 | 0.10 |
| 21 | 0.70 |
| 22 | 0.17 |
| 23 | 0.10 |
| 24 | 0.11 |
| 25 | 0.08 |
| 26 | 0.09 |
| 27 | 0.41 |
| 28 | 0.43 |
| 29 | 0.12 |
| 30 | 0.10 |
| 31 | 0.97 |
| 32 | 0.17 |
| 33 | 0.57 |
| 34 | 0.73 |
| 35 | 0.15 |
| 36 | 0.72 |
| 37 | 0.08 |
| 38 | 0.81 |
| +Control | 1.50 |

The complete disclosure of all patents, patent applications, and publications, and electronically available materials cited herein are incorporated by reference. The foregoing detailed description and examples have been given for clarity of understanding only. No unnecessary limitations are to be understood therefrom. In particular, while theories may be presented describing operation of the invention, the inventors are not bound by theories described herein. The invention is not limited to the exact details shown and described, for variations obvious to one skilled in the art will be included within the invention defined by the claims.

What is claimed is:

1. A method of producing an IgG type monoclonal antibody, comprising:
providing B-cells obtained from a transgenic non-human mammal whose genome comprises a disruption of an Act1 gene;
contacting the B cells with an antigen under conditions which result in formation of an antibody specific to the antigen by the B cells;
fusing the B cells with one or more immortal cells, to produce one or more hybridomas which express IgG type monoclonal antibodies that specifically bind to the antigen;
selecting a hybridoma which expresses an antibody specific to the antigen; and
obtaining monoclonal antibody produced by the selected hybridoma.

2. The method of claim 1, the B cells being present in a spleen cell isolate.

3. The method of claim 1, wherein the monoclonal antibodies are humanized.

4. The method of claim 1, the disruption of an Act1 gene comprising a homozygous disruption of an Act 1 gene.

5. The method of claim 1, the mammal comprising a mouse.

6. The method of claim 1, the step of selecting a hybridoma comprising the use of an immunoassay.

7. The method of claim 6, the immunoassay comprising an ELISA assay.

8. The method of claim 1, the step of fusing the B cells with an immortal cell comprising the use of a PEG polymer complex.

9. The method of claim 1, the immortal cell comprising a mouse myeloma cell.

10. The method of claim 9, the mouse myeloma cell comprising a SP2/0 mouse myeloma cell.

11. The method of claim 1, wherein the antigen is selected from the group consisting of human pathogens, allergens, bacteria, toxins, mycoplasma, fungi and viruses.

12. The method of claim 1, the step of contacting the B cells with the antigen comprising administering HCS (hybridoma cloning supplement) to the B cells.

13. The method of claim 1, further comprising the step of isolating the B cells from the transgenic non-human mammal.

14. The method of claim 1, wherein the disruption of an Act1 gene comprises a deletion of exon 2 of the Act1 gene.

15. A kit for producing an IgG monoclonal antibody specifically directed to an antigen in vitro, comprising:
B cells from a transgenic non-human mammal whose genome comprises a disruption of an Act1 gene;
immortal cells;
reagents necessary for contacting the B cells with the antigen;
reagents necessary for fusing the B cells with an immortal cell; and
a package for holding the B cells, the immortal cells, and the reagents.

16. The kit of claim 15, wherein the kit further comprises instructions for using the kit to carry out a method of producing a monoclonal antibody specifically directed to an antigen using the B cells, the immortal cells, and the reagents.

17. The kit of claim 15, wherein the kit further comprises reagents necessary for maintaining the selected hybridoma under conditions in which the monoclonal antibody is expressed.

18. The kit of claim 15, the mammal comprising a mouse.

19. The kit of claim 15, the B cells being present in a spleen cell isolate.

20. The kit of claim 15, the immortal cells comprising SP2/0 mouse myeloma cells.
